(19) United States
(12) Patent Application Publication
(10) Pub. No.: US 2023/0309515 A1
(43) Pub. Date: Oct. 5, 2023

(54) STEREOSCOPIC ECOLOGICAL POULTRY RAISING MECHANISM

(71) Applicant: MANUKA (XIAMEN) BIOTECH., LTD, Xiamen (CN)

(72) Inventor: Zhouwen LAI, Xiamen (CN)

(21) Appl. No.: 18/129,916

(22) Filed: Apr. 3, 2023

(30) Foreign Application Priority Data
Apr. 2, 2022 (CN) 202220761163.1

(51) Int. Cl. A01K 31/17; A01K 67/02; A01K 31/04

(52) U.S. Cl. CPC A01K 31/17; A01K 67/02; A01K 31/04

(57) ABSTRACT

The present application relates to the field of poultry raising, specifically to a stereoscopic ecological poultry raising mechanism. The present application provides a poultry raising mechanism, which is provided with a paddling area, a transition area connected with the paddling area, and an egg laying area connected with the transition area. The poultry raising mechanism of the present application satisfies the nature of poultry to play with water, and produced duck eggs have light offensive smell; and additionally, the breakage rate of poultry eggs can be reduced. A poultry raising apparatus provided by the present application adopts a stereoscopic multilayer raising way, which makes full use of the land resource, and reduces the production cost.

### CROSS-REFERENCE TO RELATED APPLICATIONS

This application claims priority to Chinese Patent Application 202220761163.1, filed on Apr. 2, 2022, which is incorporated herein by reference.

### FIELD

The present application relates to the field of poultry raising, specifically to a stereoscopic ecological poultry raising mechanism.

### BACKGROUND

Egg-laying ducks are waterfowl and are characterized by their preference for water and dryness, cold tolerance, and fear of heat and humidity. However, traditional modes of raising egg-laying ducks in natural water, which are dominant in China, have outstanding environmental and epidemic prevention defects. For example, the discharge of water containing duck excrement into the natural water is likely to cause pollution; furthermore, in the traditional modes of raising ducks, a plurality of households generally share a natural water area, which cannot be separated from each other and therefore cannot take biological safety measures, making it easy for diseases such as duck plague, duck Tembusu virus in ducks and the like to occur or spread.

At present, the “river chief system” is implemented in different places in China; and the “river chief system” takes the main tasks of protecting water resources, preventing water pollution, improving water environments and restoring water ecology, comprehensively establishes a four-level river chief system of the province, city, county and township, and builds a coordinated and orderly river and lake management and protection mechanism with clear responsibilities, strict supervision and strong protection to provide institutional guarantee for maintaining healthy life of rivers and lakes and realizing the sustainable utilization of functions of the rivers and lakes.

Therefore, flocks of egg-laying ducks are rarely seen in the natural water. The government encourages the dry raising of egg-laying ducks.

However, the dry raising of the egg-laying ducks has the problem that it goes against the nature of animals to play with water, and the duck eggs have a strong offensive smell. At the same time, in the existing mode of raising egg-laying ducks, the raising density of a sports ground is relatively low, leading to a waste of land resources and an increase in cost.

### SUMMARY

The present application provides a poultry raising mechanism.

The present application provides an integrated poultry raising apparatus.

The present application provides a poultry raising system.

The present application provides an application of the poultry raising mechanism in raising poultry.

In order to overcome at least some of the problems in the prior art, in an aspect, the present application provides a poultry raising mechanism.

In some implementations, the poultry raising mechanism is provided with a paddling area, a transition area connected with the paddling area, and an egg laying area connected with the transition area.

In some implementations, a junction between the egg laying area and the transition area is at least partially provided with a partition.

In some implementations, a junction between the egg laying area and the transition area is provided with a gate.

In some implementations, the gate is an automatic gate. The automatic gate can rise or fall according to the poultry species, production management, egg-laying habits, epidemic prevention requirements, seasons and weather changes.

In some implementations, the egg laying area is provided with separated units.

In some implementations, the egg laying area is provided with a bearing mechanism, and an upper surface of the bearing mechanism forms a structure arranged horizontally towards one end away from the transition area.

In some implementations, the upper surface of the bearing mechanism forms a structure inclining downwards towards one end away from the transition area.

In some implementations, the bearing mechanism is a hollowed-out structure.

In some implementations, the bearing mechanism is provided with a plurality of columnar bodies; and the columnar bodies extend downwards along one end away from the transition area, and the upper surface of the bearing mechanism is formed by upper surfaces of the columnar bodies.

In some implementations, the columnar body is a hollow tubular structure.

In some implementations, the columnar bodies are parallel to each other.

In some implementations, the columnar bodies are perpendicular to a connection line between the egg laying area and the transition area.

In some implementations, the bearing mechanism is also provided with at least one cross bar; and the cross bar is located below the columnar bodies and connected with lower surfaces of the columnar bodies.

In some implementations, the cross bar is perpendicular to the columnar body.

In some implementations, an egg collection area is formed at one end of the bearing mechanism away from the transition area, and the egg collection area or an area close to the egg collection area is provided with a shading piece.

In some implementations, the shading piece includes a shading canopy, a shading curtain, a shading plate, shading tiles, a shading membrane and a shading net.

In some implementations, the shading piece is a shading canopy.

In some implementations, one end of the bearing mechanism close to the transition area, and/or the transition area is provided with a light source.

In some implementations, the light source is arranged at the top.

In some implementations, the poultry raising mechanism is also provided with an excrement holding area, and the excrement holding area is located below the bearing mechanism.

In some implementations, the excrement holding area is provided with an excrement holding plate.

In some implementations, the excrement holding area is provided with an excrement holding belt.

In some implementations, the excrement holding plate is provided with an excrement holding belt.

In some implementations, the excrement holding plate is arranged horizontally.

In some implementations, the excrement holding area is provided with an automatic excrement scraping apparatus.

In some implementations, the excrement scraping apparatus is provided with excrement scraping plate.

In some implementations, the transition area is provided with at least one portion of inclined area, and the inclined area extends upwards from one end close to the paddling area to the other end.

In some implementations, the transition area is provided with a drinking area and a feeding area.

In some implementations, two sides of the transition area are provided with the drinking area and the feeding area respectively along an inclination direction.

In some implementations, the drinking area is a drinking pool, and the feeding area is a trough.

In some implementations, at least one portion of the paddling area is located in an open environment.

In some implementations, the paddling area is provided with a reservoir, and the bottom of the reservoir extends downwards from one end close to the transition area to the other end.

In some implementations, the bottom of the reservoir is a concave arc surface, and the bottom of the reservoir is connected with the transition area.

In some implementations, a length of the paddling area is 2-5 m, a width is 1-3 m and a height is 0.2-2 m; the length of the transition area along the inclination direction is 0.5-2, and the transition area has the same width as the paddling area; and the length of the egg laying area is 2-5 m, and the egg laying area has the same width as the paddling area. In some implementations, the length, width and height of the paddling area, the length of the transition area along the inclination direction and the length of the egg laying area are unnecessary to limit specifically, and can be increased correspondingly according to building dimensions.

In some implementations, the length of the paddling area is 2.5-4.5 m, the width is 1.8-2.2 m, and the height is 0.4-1.2 m; the length of the transition area along the inclination direction is 0.8-1.2 m; and the length of the egg laying area is 3.4-3.8 m.

In an aspect, the present application provides an integrated poultry raising apparatus, which is provided with a plurality of poultry raising mechanisms.

In some implementations, the plurality of poultry raising mechanisms are connected through fences, partition plates, diaphragms, barricades or partition nets. In some implementations, the integrated poultry raising apparatus includes a plurality of partition layers, and each partition layer includes a plurality of poultry raising mechanisms.

In some implementations, the number of partition layers is 2-10.

In some implementations, the number of partition layers is 5-10.

In some implementations, the number of partition layers is 6-9.

In some implementations, a height of the partition layer is 1.0-3.5 m, preferably 1.5-3.0 m, and more preferably 1.8-2.5 m.

In some implementations, in the plurality of partition layers, the paddling area of the poultry raising mechanism located below is provided with an extension area with at least one part not being covered by the upper layer mechanism in a vertical direction.

In some implementations, the length of the extension area that is not covered by the upper layer mechanism in the vertical direction is the length that is gradually increased by an equal distance or unequal distance of 1-10 m. For example, the length of the extension area that is not covered by the upper layer mechanism in the vertical direction may be 1 m, 2 m, 3 m, 4 m, 5 m, 6 m, 7 m, 8 m, 9 m and 10 m, may also be 5 m, 10 m, 15 m, 20 m, 25 m and 30 m, and may also be 3 m, 6 m, 10 m, 15 m, 17 m, 19 m, 25 m, 28 m and 30 m, which may be determined according to the actual building requirement.

In an aspect, the present application provides a poultry raising system, which includes the integrated poultry raising apparatus, and further includes a ventilation apparatus.

In some implementations, the ventilation apparatus adopts positive-pressure ventilation. In some implementations, the positive-pressure ventilation refers to that air of a specified air source is delivered by a fan to a specified position of a poultry house through an air pipe; and the air pressure in the air pipe is higher than the air pressure in the poultry house.

In some implementations, the air source of the ventilation apparatus is selected from basements, and/or caves and facilities with controllable temperature and humidity.

In some implementations, the ventilation apparatus is provided with a blower, and the blower draws air into the system through buried pipes and/or underground tunnels.

In some implementations, the ventilation apparatus is provided with a main air pipe, the main air pipe is located above the egg laying area, and herringbone-type, claw-type, or O-type branch pipes draws the air in a forward direction.

In some implementations, the main air pipe is hung along a lower edge of the excrement holding plate.

In some implementations, the main air pipe is hung along the top of the system.

In an aspect, the present application provides application of the poultry raising mechanism in raising poultry.

In some implementations, the poultry is selected from chicken, ducks and geese.

In some implementations, the poultry is ducks.

In some implementations, the poultry is egg-laying ducks.

In some implementations, the present application has the following characteristics:

1) The nature of the egg laying poultry to play with water is satisfied, and the produced eggs have light offensive smell.

2) By adopting the stereoscopic multilayer raising way, the land resource is used sufficiently, and the production cost is reduced.

3) By using the characteristics that the egg laying poultry prefers to lay eggs in a shade, the egg laying poultry lays eggs nearby the egg laying area, so that the breakage rate of the duck eggs is reduced.

### BRIEF DESCRIPTION OF THE DRAWINGS

FIG. 1 is a side sectional view of a single poultry house in a poultry raising mechanism according to embodiment 1.

FIG. 2 is a top view of a bottom of the single poultry house in the poultry raising mechanism according to the embodiment 1.

FIG. 3 is an enlarged detail view of a bearing platform of an egg laying area of the single poultry house in the poultry raising mechanism according to the embodiment 1;

FIG. 3 shows an enlarged view of an area A in FIG. 2, and shows a plurality of parallel columnar bodies 1 in a hollowed-out structure, and a plurality of cross bars vertically connected with the parallel columnar bodies; and FIG. 3 is a top view.

FIG. 4 is a side sectional view of the single poultry house in the poultry raising mechanism according to an implementation, and in the implementation, a light source 4 is located above a gate.

FIG. 5 is a side sectional view of the single poultry house in the poultry raising mechanism according to an implementation, and in the implementation, the light source 4 and air pipe branch pipes 9 are also arranged above the egg laying area.

FIG. 6 is an overall side sectional view of a four-layer poultry house in an implementation in the embodiment 3.

FIG. 7 is an overall side sectional view of the four-layer poultry house in an implementation in the embodiment 3.

FIG. 8 is an enlarged detail view of a heat exchanging net 19 of the four-layer poultry house in an implementation in the embodiment 3.

FIG. 9 is an overall side sectional view of a two-side eight-layer poultry house in an implementation in the embodiment 4.

FIG. 10 is a top view of a bottom of the two-side eight-layer poultry house in an implementation in the embodiment 4.

FIG. 11 is an enlarged detail view of a bearing platform of an egg laying area of a single poultry house of the two-side eight-layer poultry house in the poultry raising mechanism according to the embodiment 4; FIG. 11 shows an enlarged view of an area C in FIG. 10, and shows a plurality of parallel columnar bodies 1 in a hollowed-out structure, and a plurality of cross bars vertically connected with the parallel columnar bodies; and FIG. 11 is a top view.

FIG. 12 is an overall side sectional view of the two-side eight-layer poultry house in an implementation in the embodiment 4.

FIG. 13 is an enlarged detail view of a heat exchanging net 19 of the two-side eight-layer poultry house in an implementation in the embodiment 4.

In all accompanying drawings, the same reference numerals indicate the same technical features.

Reference numerals: 1, columnar body; 2, shading piece; 3, egg collection area; 4, light source; 5, excrement holding plate; 6, gate; 7, reservoir; 8, transition area; 9, air pipe branch pipe; 10, canopy; 11, auger conveyor; 12, manure pit; 13A, 13B, drinking pool; 14A, 14B, trough; 15, underground water; 16, water temperature regulating pool; 17, circulating water pump; 18, circulating water inlet pipe; 19, heat exchanging net; 20, circulating water outlet pipe; 21, outlet water collection trough; 22A, 22B, air inlet pipe; 23A, 23B, air supply pipe; 1901, water inlet; 1902, metal heat conducting wire.

### DETAILED DESCRIPTION

Technical solutions of the present application are further described below through specific embodiments, and the specific embodiments are not construed as limitations to the protection scope of the present application. Non-essential changes and adjustments made by others according to the concept of the present application still fall within the protection scope of the present application.

The above description is provided with reference to the accompanying drawings, which is conducive to a comprehensive understanding of various embodiments of the present application as defined by the claims. Various specific details are conducive to the understanding, but these details should be construed as exemplary only. Accordingly, those ordinary skilled in the art should recognize that changes and improvements can be made to the various embodiments described herein without departing from the scope of the present application as defined by the appended claims. Furthermore, for clarity and simplicity, descriptions of well-known functions and structures may be omitted.

Terms and expressions used in the following description and claims are not limited to written meanings, but are only used by the inventor to allow a clear and consistent understanding of the present application. Accordingly, it is obvious to those skilled in the art that the following descriptions of various embodiments of the present application are provided for the purpose of explanation only, and are not intended to limit the present application as defined by the appended claims.

In the description of the present application, it should be noted that the terms “center”, “upper,” “lower,” “front,” “rear,” “left,” “right”, “vertical”, “horizontal”, “top”, “bottom”, “inside”, “outside”, and the like indicating the orientation or positional relationship are based on the orientation or positional relationship shown in the drawings, and are only used for convenience in describing the present application and simplifying the description, rather than indicating or implying that specific devices or elements must have a specific orientation and be constructed and operated in a specific orientation. Therefore, the terms cannot be understood as limitations to the present application. In the description of the present application, the term “a plurality of” means two or more, unless otherwise specified.

Furthermore, technical characteristics involved in different embodiments of the present application described below can be combined if there is no contradiction.

It should be understood that singular forms “a”, “an”, “the” and “said” include plural referents unless otherwise specified clearly in the context. The expression “or” in the present application includes any or all combinations of words listed together. For example, “A or B” may include A or B, or may include both A and B. It should also be understood that the term “and/or” as used herein refers to and includes any or all possible combinations of one or more associated listed items.

Throughout the specification and claims of the present application, the words “comprising” and “including” and variations of words, such as “includes” and “comprises” mean “includes but not limited to”, and are not intended to (and may not) exclude other components, integers or steps.

In the description of embodiments of the present application, it should be noted that unless otherwise specified and limited, the terms “arranged”, “provided with” and “connection” should be understood in a broad sense, for example, “connection” may be fixed connection, detachable connection or integrated connection, may also be mechanical connection, electrical connection, direct connection or indirect connection through an intermediate medium, and may also be internal communication of two elements. For those ordinary skilled in the art, the specific meaning of the above terms in the present application may be understood in specific circumstances.

It should be noted that the same reference numerals and letters indicate the same items in the following drawings, so once an item is defined in one drawing, the item does not need to be further defined and explained in the following drawings.

### Embodiment 1

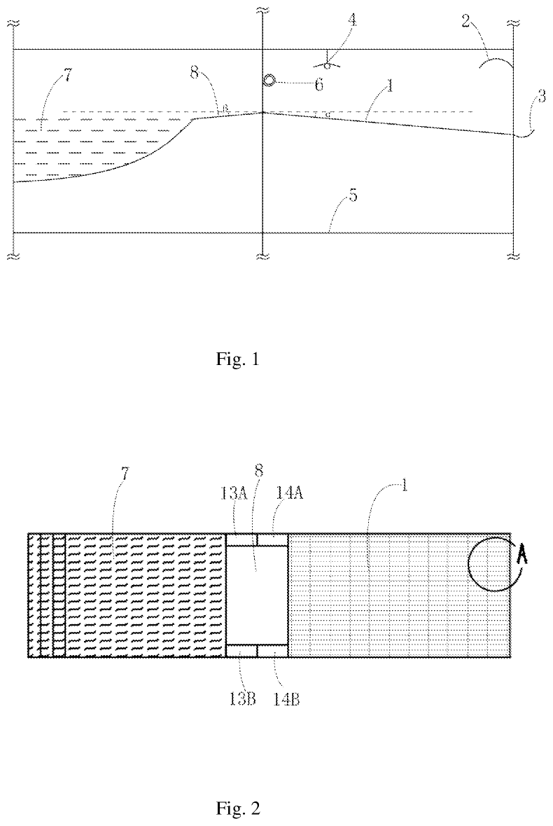

As shown in FIG. 1, an embodiment of the present application provides an egg-laying duck raising mechanism, which includes a paddling area, a transition area, and an egg laying area. The paddling area is connected with one end of the transition area, and the other end of the transition area is connected with the egg laying area. The paddling area and the transition area are half-open environments, the egg laying area is an indoor environment, the egg laying area and the transition area are separated by a wall, and the wall is provided with an automatic rolling gate 6. The automatic gate is opened before night falls, ducks are transferred indoors, the automatic gate falls automatically during day, and the ducks are rushed into the paddling area and the transition area for swimming and outdoor feeding. The automatic rolling gate can rise or fall according to the poultry species, production management, egg-laying habits, epidemic prevention requirements, seasons and weather change.

Water is stored in a reservoir 7 of the paddling area and can be used for egg-laying ducks to swim and play during the day, and some egg-laying ducks may lay eggs in the reservoir. The water in the reservoir is changed every day to keep the water fresh and clean. One end of the paddling area away from the transition area 8 is partially open, which may ensure that the ducks receive sunlight.

As shown in FIG. 2, the transition area 8 can be used for the egg-laying ducks to roost and tidy feathers during the day, and drinking pools 13A and 13B and troughs 14A and 14B which are arranged at two sides of the transition area can be used for the egg-laying ducks to drink water and take food. The drinking pools can automatically supplement the water by induction, and the troughs can supply food automatically, thereby ensuring the precise feeding. When the egg-laying ducks go ashore from the reservoir, they can drip dry in the transition area, so that when the egg-laying ducks enter the poultry house, the interior of the poultry house can be kept dry as far as possible.

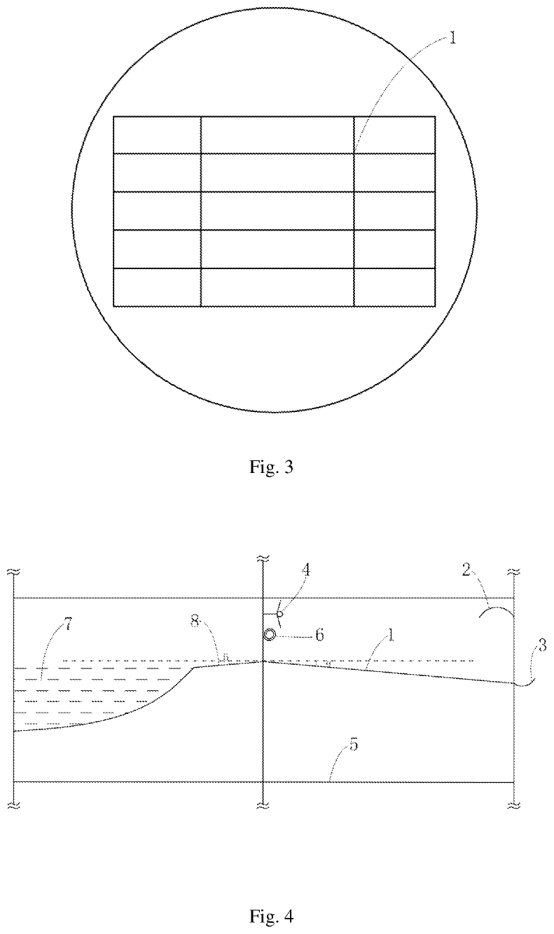

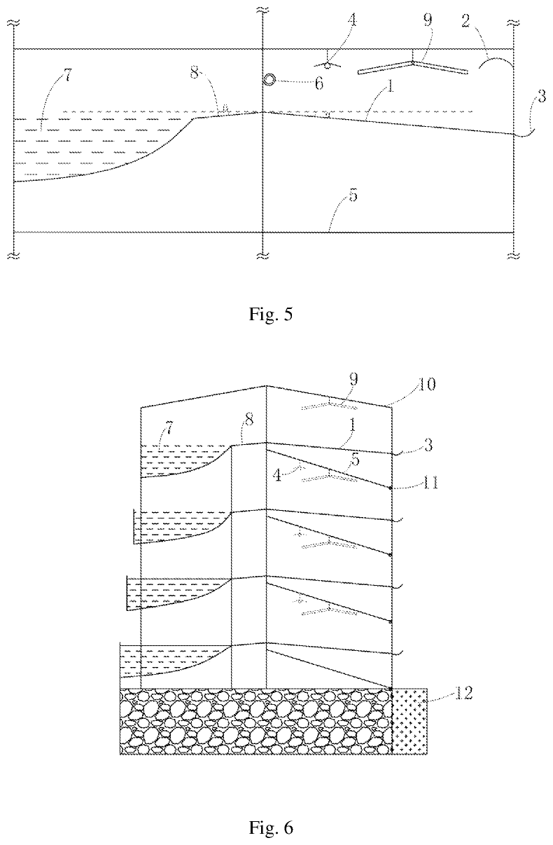

The egg laying area is connected with the transition area; and as an indoor environment, the egg laying area includes an inclined bearing platform which can be used for the egg-laying ducks to roost and lay eggs at night. One end of the bearing platform close to the transition area 8 is higher than the other end away from the transition area; and one end away from the transition area is an egg collection area, so that after the egg-laying ducks lay the eggs, the duck eggs can be collected in the egg collection area 3. The bearing platform includes a hollowed-out structure, and a detail of the bearing platform is as shown in FIG. 3. The hollowed-out structure is formed by a plurality of parallel columnar bodies 1, and specifically formed by hollow stainless steel pipes, and a plurality of cross bars connected with the stainless steel pipes vertically. A plane formed by axes of the cross bars is not the same plane as the plane formed by the axes of the stainless steel pipes; and the cross bars are located below the stainless steel pipes, and each cross bar is connected with the lower surfaces of the plurality of stainless steel pipes. The upper surface of the bearing platform is formed by a plane formed by the upper surfaces of the plurality of the parallel stainless steel pipes, the egg-laying ducks can stand on the bearing platform, and a width of a clearance among the stainless steel pipes shall ensure that duck feet may not fall into the clearance and get stuck. The cross bars play a role in supporting and fixing the stainless steel pipes. The axis of the stainless steel pipe is perpendicular to a wall where the egg laying area and the transition area are connected in a top view. After the egg-laying eggs lay eggs on the upper surface of the bearing platform, the duck eggs may be rolled to a lowest place of the hollowed-out structure along the clearance among the stainless steel pipes.

The egg laying area also includes an excrement holding plate 5; the excrement holding plate is located below the bearing platform and is arranged horizontally, and liquid dung produced by the egg-laying ducks in the egg laying area may finally drop onto the excrement holding plate through the hollowed-out structure. Furthermore, the excrement holding plate is also provided with an automatic excrement scraping apparatus, which may automatically clear the liquid dung on the excrement holding plate.

Dimensions of each part in the egg-laying duck raising mechanism provided by the present embodiment are as follows:

A length of the paddling area is 2.6 m, a width is 2 m, and a height is 1 m; the length of the transition area along an inclination direction is 1 m, and the transition area has the same width as the paddling area, which is 1 m; and the length of the egg laying area is 3.6 m, and the egg laying area has the same width as the paddling area, which is 1 m.

### Embodiment 2

Another implementation of the present application is as shown in FIG. 2-FIG. 3, and optionally, the egg collection area is also provided with a shading canopy 2. The top of a wall side of a wall close to the egg laying area is provided with a light source 4. When the ducks enter the egg laying area at night, after the light source is turned on, the ducks tend to gather under the shading canopy and thus lay the eggs nearby the egg collection area.

### Embodiment 3

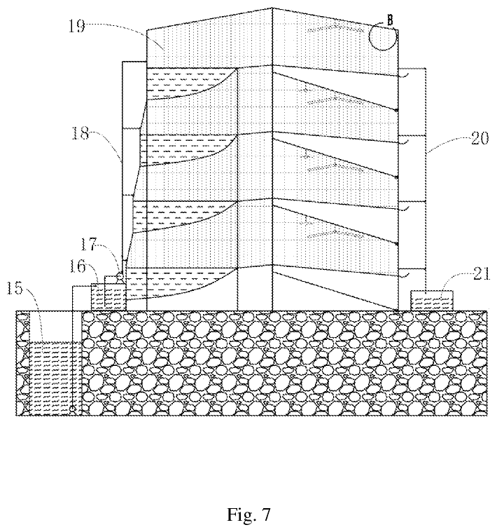

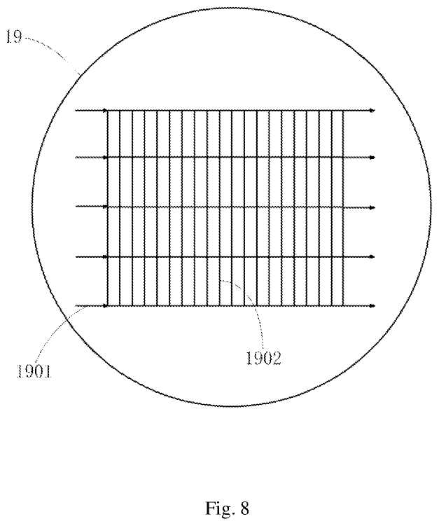

Another implementation of the present application is as shown in FIG. 6 and FIG. 7. The present application provides a four-layer poultry house, which includes walls (6 walls in total), a canopy 10 arranged on the walls, and a ventilation apparatus; and a plurality of partition layers are successively arranged in the poultry house from top to bottom, and each partition layer includes a plurality of poultry raising mechanisms as described in the embodiment 2. The paddling area of the laying duck raising mechanism is arranged towards the outside of a duck shed, and an egg laying end is arranged towards the inside of the duck shed. The reservoir of the egg-laying duck raising mechanism on each layer extends outwards by 20 cm from top to bottom, so that the ducks on each layer can receive the sunlight.

The ventilation apparatus includes a blower, an underground tunnel, a buried pipeline, a main air pipe, and branch pipes. The underground tunnel, the buried pipeline, the main air pipe, and the branch pipes are communicated. The main air pipe of the non-top layer is hung at a lower edge of the excrement holding plate of each layer of egg-laying duck raising mechanism, and the main air pipe of the top layer is hung at the lower edge of the canopy and located above the egg laying area of the top poultry raising mechanism. The branch pipes 9 are in herringbone type. The ventilation apparatus adopts positive-pressure ventilation, that is, the blower may blow the air from basements and caves into the poultry house through the underground tunnels and pipelines, and the blown air flows by the main air pipe and then flows by the branch pipes, so that the air is drawn into the egg laying area to realize the ventilation.

### Embodiment 4

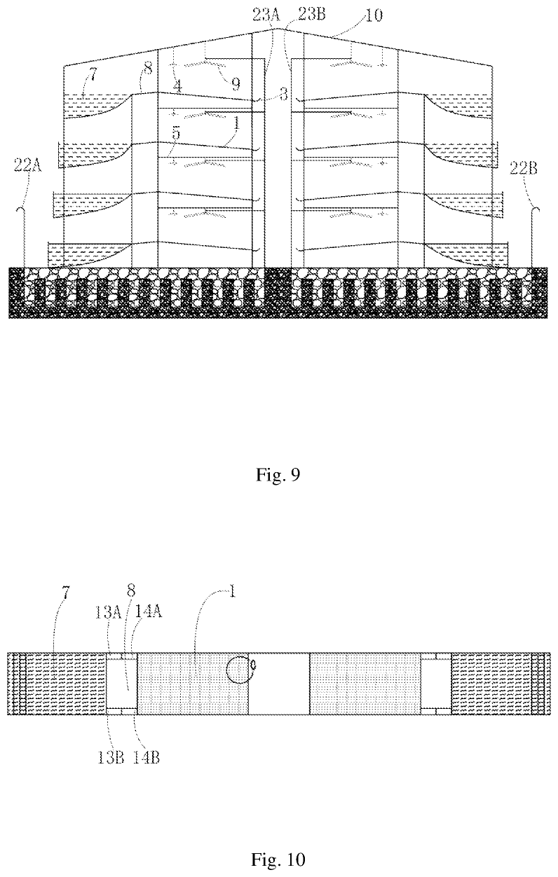

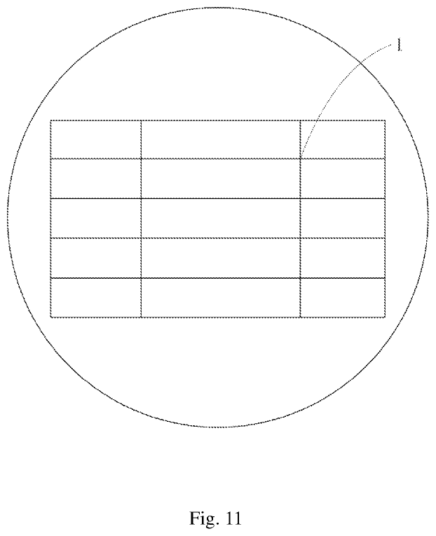

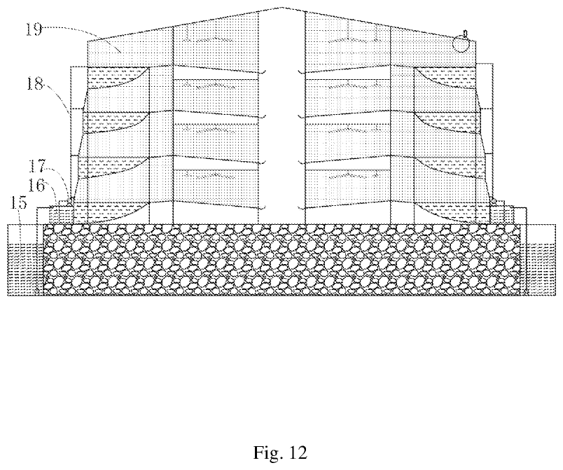

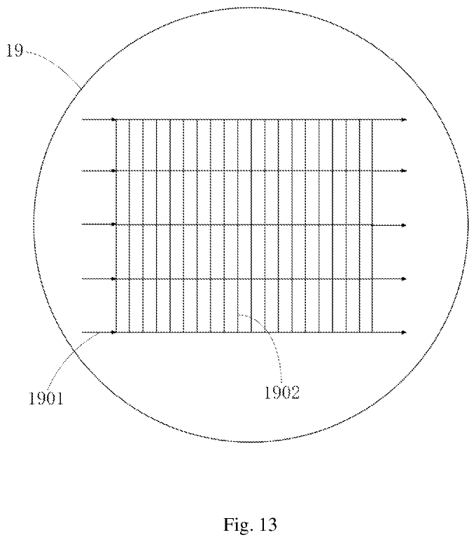

Another implementation of the present application is as shown in FIG. 9 and FIG. 12. The present application provides a two-side eight-layer poultry house, which includes walls (6 walls in total), a canopy 10 arranged on the walls, and a ventilation apparatus; and a plurality of partition layers are successively arranged in a duck shed from top to bottom, and each partition layer includes a plurality of poultry raising mechanisms as described in the embodiment 2, wherein in each partition layer, the poultry raising mechanisms are symmetrically arranged in two rows. The paddling area of the poultry raising mechanism is arranged towards outside of the duck shed, and an egg laying end is arranged towards the inside of the duck shed. The reservoir of the poultry raising mechanism on each layer extends outwards by 20 cm from top to bottom, so that the poultry on each layer can receive the sunlight.

The ventilation apparatus includes a blower, an underground tunnel, a buried pipeline, a main air pipe, and branch pipes. The underground tunnel, the buried pipeline, the main air pipe, and the branch pipes are communicated. The main air pipe of a non-top layer is hung at a lower edge of the excrement holding plate of each layer of poultry raising mechanism, and the main air pipe of the top layer is hung at the lower edge of the canopy and located above the egg laying area of the top poultry raising mechanism. The branch pipes 9 are in herringbone type. The ventilation apparatus adopts positive-pressure ventilation, that is, the blower may blow the air from basements and caves into the poultry house through the underground tunnels and pipelines, and the blown air flows by the main air pipe and then flows by the branch pipes, so that the air is drawn into the egg laying area to realize the ventilation.

What is claimed is:

1. A poultry raising mechanism, being provided with a paddling area, a transition area connected with the paddling area and an egg laying area connected with the transition area.

2. The poultry raising mechanism according to claim 1, wherein a junction between the egg laying area and the transition area is at least partially provided with a partition.

3. The poultry raising mechanism according to claim 1, wherein the egg laying area is provided with a bearing mechanism, and an upper surface of the bearing mechanism forms a structure arranged horizontally towards one end away from the transition area.

4. The poultry raising mechanism according to claim 3, wherein an egg collection area is formed at one end of the bearing mechanism away from the transition area, and the egg collection area or an area close to the egg collection area is provided with a shading piece.

5. The poultry raising mechanism according to claim 3, wherein the poultry raising mechanism is also provided with an excrement holding area, and the excrement holding area is located below the bearing mechanism.

6. The poultry raising mechanism according to claim 1, wherein the transition area is provided with at least one portion of inclined area, and the at least one portion of inclined area extends upwards from one end of the transition area close to the paddling area to a second end of the transition area.

7. The poultry raising mechanism according to claim 1, wherein a length of the paddling area is 2-5 m, a width of the paddling area is 1-3 m, and a height of the paddling area is 0.2-2 m, a length of the transition area along an inclination direction is 0.5-2 m, the transition area has a same width as the width of the paddling area, a length of the egg laying area is 2-5 m, and the egg laying area has a same width as the width of the paddling area.

8. An integrated poultry raising apparatus, being provided with a plurality of poultry raising mechanisms according to claim 1, wherein
the plurality of poultry raising mechanisms are connected through fences, partition plates, diaphragms, barricades or partition nets.

9. A poultry raising system, comprising the integrated poultry raising apparatus according to claim 8, and further comprising a ventilation apparatus.

10. An application of the poultry raising mechanism according to claim 1 in raising poultry.

11. The poultry raising mechanism according to claim 2, wherein:
the partition comprises an automatic gate, and
the egg laying area is provided with separated units.

12. The poultry raising mechanism according to claim 3, wherein:
the upper surface of the bearing mechanism forms a structure inclining downwards towards one end away from the transition area,
the bearing mechanism is a hollow tubular structure,
the bearing mechanism is provided with a plurality of columnar bodies,
the plurality of columnar bodies extend downwards along the one end away from the transition area,
the upper surface of the bearing mechanism is formed by upper surfaces of the plurality of columnar bodies,
the plurality of columnar bodies are parallel to each other,
the plurality of columnar bodies are perpendicular to a connection line between the egg laying area and the transition area,
the bearing mechanism is also provided with at least one cross bar,
the cross bar is located below the plurality of columnar bodies and connected with lower surfaces of the plurality of columnar bodies, and
the cross bar is perpendicular to the plurality of columnar bodies.

13. The poultry raising mechanism according to claim 4, wherein:
the shading piece is a shading canopy,
at least one of one end of the bearing mechanism close to the transition area or the transition area is provided with a light source, and
the light source is arranged at a top of the at least one of the one end of the bearing mechanism close to the transition area or the transition area.

14. The poultry raising mechanism according to claim 5, wherein:
the excrement holding area is provided with an excrement holding plate,
the excrement holding plate is provided with an excrement holding belt,
the excrement holding plate is arranged horizontally,
the excrement holding area is provided with an automatic excrement scraping apparatus, and
the excrement scraping apparatus is provided with excrement scraping plate.

15. The poultry raising mechanism according to claim 6, wherein:
the transition area is provided with a drinking area and a feeding area,
two sides of the transition area are provided with the drinking area and the feeding area respectively along an inclination direction,
the drinking area is a drinking pool,
the feeding area is a trough,
at least one portion of the paddling area is located in an open environment,
the paddling area is provided with a reservoir,
a bottom of the reservoir extends downwards from the one end of the transition area close to the transition area to the second end of the transition area,
the bottom of the reservoir is a concave arc surface, and
the bottom of the reservoir is connected with the transition area.

16. The poultry raising mechanism according to claim 7, wherein:
the length of the paddling area is 2.5-4.5 m,
the width of the paddling area is 1.8-2.2 m,
the height of the paddling area is 0.4-1.2 m,
the length of the transition area along the inclination direction is 0.8-1.2 m, and
the length of the egg laying area is 3.4-3.8 m.

17. The integrated poultry raising apparatus according to claim 8, wherein
the integrated poultry breeding apparatus comprises a plurality of partition layers,
each partition layer is provided with a plurality of the plurality of poultry raising mechanisms,
a number of partition layers in the plurality of partition layers is 6-9,
two or more poultry raising mechanisms of the plurality of poultry raising mechanisms are arranged symmetrically in the partition layer,
a height of the partition layer is 1.0-3.5 m,
in the plurality of partition layers, the paddling area of the poultry raising mechanism located below is provided with an extension area with at least one part not being covered by an upper layer mechanism in a vertical direction, and
a length of the extension area that is not covered by the upper layer mechanism in the vertical direction is a length that is gradually increased by an equal distance or unequal distance of 1-10 m.

18. The poultry raising system according to claim 9, wherein:
the ventilation apparatus adopts positive-pressure ventilation,
an air source of the ventilation apparatus is selected from at least one of basements, caves, or facilities with controllable temperature and humidity,
the ventilation apparatus is provided with a blower,
the blower draws air into the poultry raising system through at least one of buried pipes or underground tunnels,
the ventilation apparatus is provided with a main air pipe,
the main air pipe is located above the egg laying area,
herringbone-type, claw-type, or O-type branch pipes draw the air in a forward direction, and
one of:
the main air pipe is hung along a lower edge of an excrement holding plate, or
the main air pipe is hung along a top of the poultry raising system.

19. The application of the poultry raising mechanism according to claim 10, wherein the poultry is egg-laying ducks.
